(19) United States
(12) Patent Application Publication
(10) Pub. No.: US 2005/0095266 A1
(43) Pub. Date: May 5, 2005

(54) Surface treatment by photopolymerisation to obtain biocidal properties

(76) Inventors: Alain Perichaud, Marseille (FR); Monica Arnautu, Marseille (FR)

Correspondence Address: DENNISON, SCHULTZ, DOUGHERTY & MACDONALD, 1727 KING STREET, SUITE 105, ALEXANDRIA, VA, 22314, US

(21) Appl. No.: 10/496,792

(22) PCT Filed: Nov. 4, 2003
(86) PCT No.: PCT/FR03/03292

(30) Foreign Application Priority Data
Nov. 8, 2002 (FR) 02/14064

(51) Int. Cl. A61F 2/00; A61J 1/00

(52) U.S. Cl. 424/423; 427/2.14

(57) ABSTRACT

The present invention relates to a process for surface treatment of a solid substrate in which photopolymerisation and covalent grafting are performed in situ on said substrate of a biocidal copolymer, wherein steps are taken in which: a) said solid substrate is put in contact with a formulation comprising: 1—at least one monomer comprising a biocidal group, 2—at least one copolymerisable compound with said biocidal monomer comprising a multifunctional monomer or oligomer mono- di- or selected amongst acrylate, epoxide or vinyl ether monomers or oligomers, 3—at least one photoprimer selected amongst radical and/or cationic photoprimers, and 4—at least one grafting agent on said substrate, and b) photocopolymerisation and covalent grafting of the copolymers obtained are carried into effect by subjecting said formulation in contact with said solid substrate to ultraviolet radiation.

The present invention relates to a process for treating the surface of a solid substrate with a view to imparting it with biocidal and especially antibacterial properties.

More particularly, the present invention relates to a process for surface treatment by photopolymerisation and covalent grafting on said solid substrate of a copolymer comprising groups having biocidal activity.

The present invention relates to solid substrates comprising said copolymer grafted to their surface, obtained by said process.

The present invention relates to treatment of solid substrates which can be used to manufacture all types of products or equipment and especially textiles, floor coverings, sanitary devices especially for communities, medical instruments and equipment.

Said solid substrate can be made from all types of materials, namely organic or inorganic materials, natural or synthetic. Materials of the plastic type, and materials based on natural polymers such as polysaccharides such as paper or wood are proposed more particularly as organic materials.

More particularly still, the present invention relates to the treatment of fibrous organic materials such as textile materials or non-woven materials, manufactured from a base of synthetic threads or fibres such as polyester, polyamide or polyacrylic threads or fibres or natural fibres, especially based on cotton or wool, or in the case of paper, cellulose fibres.

Examples of inorganic material more particularly are ceramic materials, glass or metals.

In the present description, <<biocidal activity>> is understood to mean any antimicrobial or antiseptic activity, that is, and also antibacterial activities, namely bactericidal and/or bacteriostatic, anti-fungal, anti-yeast and more particularly all types of micro-organisms, especially harmful, even pathogenic.

The previous patent by the applicant, WO98/29463, describes homopolymers exhibiting strong antimicrobial activity comprising quaternary ammoniums in a predominant quantity, constituted by an ester and/or amide resin to which quaternary ammonium salts are bound by a covalent bond, and in which the rate of quaternary ammonium is at least 80% of the mass of the polymer.

In WO98/29463, these polymers were utilised more particularly to manufacture paint, a coating applicable on any type of object for which it is necessary to guard against the risks of development of microorganisms and bacteria in particular. Said homopolymer was used by way of binder in said paints or coatings.

In this patent WO 98/29463, the homopolymers could be obtained by polymerisation of monomers comprising a quaternary ammonium group, either in organic solvent phase, or in aqueous phase. The subject is radical polymerisation, with the reaction temperature close to 80° C. being maintained.

In WO 98/29463, the polymers comprising groups of quaternary ammonium salts respond to the general formula (Ia): 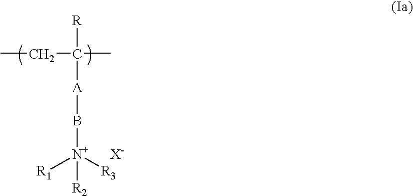
in which:
   A represents an identical or different radical 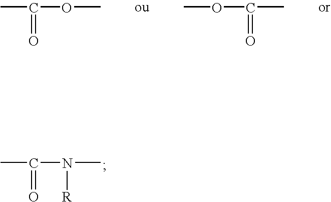
   selected amongst:
   R represents H or CH₃;
   B represents an alkylene chain in C₀-C₅, linear or branched, or an arylene or arylalkylene group;
   R₁ and R₂, which are identical or different, each represents an alkyl chain in C₁-C₅;
   R₃ represents an alkyl chain in C₈-C₂₀ or an aryl or aryl alkyl group;
   X⁻ represents an anion;
in which the rate of quaternary ammonium is greater than 1 mole/kg.

The monomers comprising a quaternary ammonium group of formula (Ia) are highly efficacious in terms of biocidal effect, but are difficult to photopolymerise and to graft onto a solid substrate.

This is why these biocidal polymers are simply deposited on the surface of the solid substrates to be coated where they benefit from relatively strong adhesion by adsorption on the surface by means of physical-chemical interaction.

However, for certain applications, the bonds of said polymers on the surface of the solid substrate are not strong and stable enough to maintain biocidal and/or biostatic activity over a prolonged period. This is the case in particular for objects which might undergo frequent washing or frequent cleaning maintenance, the antiseptic or biocidal properties having neither durability nor sufficient resistance to the conditions of use and maintenance. This is likewise the case of medical equipment such as, for example: catheters, gastric probes, blood collection scoop, for which the biocide does not have to be salted out.

FR 2 695 800 and EP 591 024 describe biocidal or antiseptic polymers comprising quaternary ammonium groups attached to a radical methacrylate or methacrylamide, said polymers being grafted on a substrate of textile fibres by radical activation under the effect of ionising radiation such as gamma radiation, or by electronic bombardment on said substrate in the presence of monomers comprising quaternary ammonium groups.

Nevertheless, use of this type of polymerisation and grafting of the compound on the substrate on an industrial scale represents a very substantial technological investment and comes with risks having a harmful effect such as radiation for personnel.

In WO 97/47696 it was attempted to polymerise monomers comprising quaternary ammonium groups of the type of monomers of formula (Ia), as described in the patent WO 98/29463, by photo activation in contact with the substrate constituted by materials which are to serve as instruments for medical use made from base materials such as polyurethane or silicon, so as to increase the adhesion of the polymer on said substrate, said polymerisation being obtained by exposure to ultraviolet radiation.

WO 97/47696 resorts to a formulation comprising 4 essential constituents, namely: a monomer comprising bactericidal quaternary ammonium groups, reticulable oligomer, especially of polyurethane diacrylate type, adhesion to the polyurethane substrate, a photopriming agent and mono or multifunctional monomers whereof the function of diluting reagent modifies the speed of polymerisation, the physical-chemical properties of the reticulated copolymer obtained and the viscosity of the formulation.

Photopolymerisation processes under UV radiation are advantageous since they are easy to implement on an industrial scale. Nevertheless, the treatment process described in WO 97/47696 is specific to substrates constituted by polyurethane and above all do not allow grafting of the polymer obtained on the solid treated substrates, but only deposit whereof the adhesion is based on the compatibility of the two polymers (polyurethane).

Similarly, in WO 00/05281 a deposit is made, especially on a textile substrate, of a biocidal product obtained by radical copolymerisation, by simple impregnation using the padding technique, followed by evaporation of the solvents. In this way the bactericidal product deposited is likely to be removed by washing or by other routine domestic techniques or dry cleaning.

EP 0955069 describes a process for treating a material by a solution in which is dissolved an <<ionic molecule>> and/or an <<ionic polymer>> which react with a precipitant agent to form, in situ, on said material an insoluble deposit, but the precipitant agent is fixed on the substrate by once again using the technique of electronic bombardment, an expensive and harmful process.

WO 93/17746 describes obtaining medical implants or catheters which have been coated with an antibiotic or with mixtures of antibiotics by simple ionic bond between the latter and the substrate.

The patent FR 2 751 882 describes several surface modification processes of different substrates by chemical or physical activation calling on classic activation techniques via hard chemical oxidation or via plasma. This patent still calls on fairly laborious chemical treatment techniques, or again on plasma techniques which imply substantial financial investments.

In U.S. Pat. No. 6,248,811 coatings of bactericidal polymers, fixed covalently on a support, which is a polysiloxane film, are prepared.

The bactericidal monomer has a specific formula R-(A)ₙ, A being an acid or sulfonic acid salt group in the embodiments and extended to other acid groups (carboxylic, sulphuric, phosphoric and phosphonic) in claim 1.

In the process in U.S. Pat. No. 6,248,811 the following successive steps are used:
synthesis of the copolymer by copolymerisation under UV treatment of bactericidal monomers and monomers sensitive to UV, and
previous activation of the substrate to be coated of the polymer by different physical treatments such as UV treatment, corona, plasma, electronic bombardment, and . . . , and
deposit in solution of the copolymer preformed on the active support followed by new physical grafting treatment, especially by UV radiation.

The procedure described is accordingly long and laborious to put into practice, implying three steps: previous synthesis of the copolymer, activation of the support and physical grafting treatment of the copolymer on an active support.

The activation processes of the support risk degrading the polymeric supports and are not applicable for any type of polymeric support other than polysiloxane. In addition, after contact of the preformed copolymer in solution and of the support, the whole must undergo physical treatment, especially being irradiated under UV for a fairly long time, to perform the grafting, such that this type of treatment cannot be carried out for any type of bactericidal monomer. In particular, the biocidal monomers of quaternary ammonium having wide activity ranges, at the same time antibacterial and anti-fungal, of formula Ia, such as described in WO 98/29463, would not support UV treatment of a duration and intensity such as described in this U.S. Pat. No. 6,248,811.

Finally, in the U.S. Pat. No. 6,248,811, since the preformed copolymer must be soluble, the grafted biocidal copolymer obtained cannot be reticulated, which limits the properties of mechanical resistance and resistance to chemical agents and other environmental conditions.

The aim of the present invention is to provide a process for surface treatment of a solid substrate, so as to obtain covalent grafting on the surface of said solid substrate, polymers comprising biocidal groups, especially quaternary ammonium, by a process, which does not require employing significant technological means such as gamma radiation or electronic bombardment.

Another aim of the present invention is to provide a process for surface treatment of a solid substrate enabling a reticulated biocidal copolymer to be grafted on the solid substrate covalently.

Another aim of the present invention is to provide a process for grafting biocidal copolymers to the surface of a solid substrate, which is simple and inexpensive to perform, at the same time providing improved coating characteristics in terms of mechanical behaviour and resistance to environmental conditions and, more particularly, obtaining possibly more significant coating thicknesses.

To achieve this, the inventors discovered that it was possible to perform grafting of a biocidal polymer especially comprising quaternary ammonium groups onto any type of solid substrate, by resorting to radical or cationic or hybrid (radical and cationic) photopolymerisation under UV radiation, subject to employing a treatment method and appropriate reagents.

More precisely, the present invention provides a treatment process for the surface of a solid substrate in which photopolymerisation and covalent grafting are carried out in situ on said substrate of a biocidal or antiseptic copolymer, wherein steps are performed in which:
a) said solid substrate is put in contact with a formulation comprising:

1—at least one monomer comprising a biocidal group,
2—at least one copolymerisable compound with said biocidal monomer comprising a mono-, di-, or multifunctional monomer or oligomer selected amongst acrylate, epoxide or vinyl ether monomers or oligomers,
3—at least one photoprimer selected amongst radical and/or cationic photoprimers, and
4—at least one grafting agent on said substrate, and
b) photocopolymerisation and covalent grafting of the copolymers obtained are carried into effect by subjecting said formulation in contact with said solid substrate to ultraviolet radiation.

According to the present invention therefore it was discovered that by using copolymerisable monomers or oligomers, appropriate reagents and appropriate grafting stimulators, it was possible to obtain, via UV treatment, polymers containing sufficient biocidal groups, especially quaternary ammonium on the one hand, and on the other hand, covalent grafting of said polymers on the substrate, in order to obtain permanently resistant biocidal properties on said treated solid substrate, without salting out of the environment.

The process according to the present invention thus produces a durable resistant biocidal effect by a process for simple surface treatment to be carried out in accordance with the aim of the present invention.

In a preferred embodiment of the process, in step a) for contacting said formulation with said substrate, the following two successive sub-steps are carried out:
a1) said solid substrate is put in contact with a first partial formulation containing said photoprimer and said grafting agent, and
a2) after drying a second partial formulation containing said biocidal monomer and said copolymerisable compound is added.

In this way, better contact of the photoprimer and the grafting agent with the substrate is assured, which improves the grafting rate of the biocidal copolymers on said substrate, as has been demonstrated according to the present invention, in the examples hereinbelow.

<<Putting in contact>> is understood to mean that a solution of said formulation is deposited on said substrate if it is a substrate exhibiting a plane surface, such as a film, a sheet or a plate, or if said substrate is impregnated with a formulation solution, it is a fibrous substrate, woven or not woven, or a thread. In the second case said contact can be realised by pulverisation of a solution of said formulation on said substrate or by soaking of said substrate in a solution of said formulation.

This preferred embodiment in which step a) of putting in contact comprises 2 sub-steps a1) and a2) is particularly advantageous for the treatment of woven or non-woven materials whereof the threads or fibres can thus be impregnated with photopriming reagents and grafting agents, thus contributing to improving the polymerisation and grafting reaction on the substrate during application of UV radiation.

In an advantageous embodiment, in step 2), ultraviolet radiation having a consigned intensity of 10 to 5000 mW/cm² wavelength between 280 and 500 nm is applied, and a filter enabling elimination of infrared radiation and irradiation of a wavelength from 360 to 500 nm is preferably used.

More particularly, in step 2) ultraviolet radiation is used for 5 to 60 seconds, preferably 10 to 30 seconds, with an intensity of 100 to 1000 mW/cm².

More particularly, after step 2 the following step is taken in which:
3) polymerisation is carried out via thermal polymerisation by drying said substrate in an oven at a temperature between 100 and 180° C.

To carry out photopolymerisation any type of UV lamp of various dimensions and strength can be used, but the concentration and the domain of UV absorption of the photoprimer utilised is taken into consideration.

Said photopriming compound can be a radical photoprimer or a cationic photoprimer. Likewise, a hybrid mechanism can be used by utilising two respectively radical and cationic photoprimers. The choice of said radical or cationic photoprimers depends on the choice of said biocidal monomers and of said copopolymerisable compounds, that is, reagent groups which they comprise according to the fact that the latter can be activated radically or cationically. In particular two respectively radical and cationic photoprimers are to be used when the formulation comprises two types of said copolymerisable compounds, photopolymerisable respectively radically and cationically.

In a preferred embodiment and according to another characteristic of the present invention, said biocidal monomer comprises a monomer comprising a group of quaternary salts responding to the formula (I) in which:

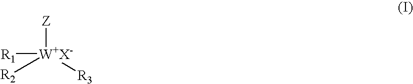

In which:
Z represents a monovalent radical selected amongst
either 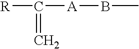
in which:
R represents —H or —CH₃
A represents: 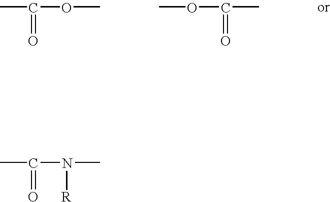
   B represents an alkylene chain in C₁-C₅, linear or branched or an arylene or arylalkylene group
   or CₙH₂ₙ₋₁(OH)₂—B—
   or HO—(B—O)ₐ—B—
in which B has the meaning given hereinabove and n can vary from 1 to 20 and a can vary from 0 to 3.
W⁺ represents a N⁺ nitrogen cation, P⁺ phosphorous or Q⁺ a saturated or unsaturated heterocycl comprising a nitrogen atom substituted by R₃, or directly bound to A or to B, and likewise able to contain in addition to quaternised nitrogen one or more hetero atoms, identical or different
R₁ and R₂ identical or different, each representing an alkyl chain in C₁-C₅ or an aryl group
R₃ represents an alkyl chain in C₃-C₂₀ or an aryl or aryl alkyl group
X⁻ represents an anion, especially halide.

Said biocidal monomer of formula (I) differs in function from the type of mechanism employed for photopolymerisation.

The biocidal monomers of formula (I) hereinabove, for which Z represents 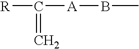
are adapted to copolymerisation by radical photopolymerisation and thus require the presence of a radical photoprimer.

The biocidal monomers of formula (I) hereinabove, for which Z represents
CₙH₂ₙ₋₁(OH)₂—B—
HO—(B—O)ₐ—B—
are adapted to copolymerisation by cationic photopolymerisation and thus require the presence of a cationic photoprimer.

For radical photopolymerisation, advantageously the monomer of the following formula (I₁) will be used: 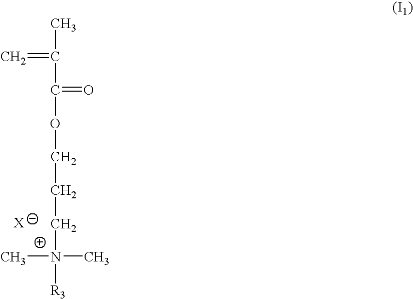
in which:
R₃ represents an alkyl chain in C₈-C₁₆, an aryl or aryl alkyl group, and
X⁻ represents an anion, especially halide

For cationic photopolymerisation, the biocidal monomer of the following formula (I₂) will advantageously be used: 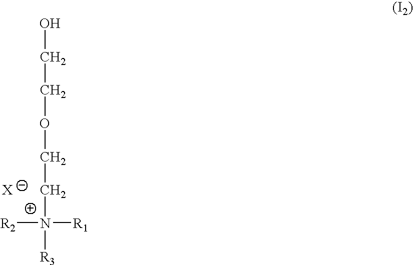
in which:
X⁻ represents an anion,
R₁ and R₂ identical or different, each represent an alkyl chain in C₁-C₅ or an aryl group,
R₃ represents an alkyl chain in C₃-C₂₀ or an aryl group

Only the monomers of formula (I₁) are described in WO98/29463.

To carry out polymerisation and grafting on the substrate of the biocidal polymer resulting from the copolymerisation of said biocidal monomer and of said copolymerisable monomer or oligomer, it is necessary to utilise a grafting agent which can be either a grafting primer for direct grafting on the substrate, or a coupling agent for indirect grafting on the substrate. “Grafting primer” is understood to mean a compound which enables active centres to be created on the support, active centres from which direct covalent chemical bonds of the substrate could be established with the biocidal polymer resulting from copolymerisation of said biocidal monomer and of said copolymerisable monomer or oligomer. “Coupling agent” is understood to mean a compound capable on the one hand of creating an intermediate covalent chemical bond between the substrate and said biocidal polymer by reaction of said coupling agent on a chemical function borne by the substrate and on the other hand by polyaddition or by polycondensation of said coupling agent to form a copolymer with said biocidal monomers and said copolymerisable compounds contained in the formulation.

The grafting agents thus result in the formation of covalent bonds between the substrate and the coating of biocidal polymer as they are capable either of substituting a hydrogen of the substrate, especially hydrogens belonging to a tertiary carbon with respect to the grafting primers, or of reacting chemically with said functional groups of the substrate and with said functional groups of said monomers and/or said polymerisable compounds of the formulation with respect to the coupling agents.

These <<grafting agents>> compounds can belong to the following categories and families:

A. Grafting Primers.

These grafting primers can be activated radically exclusively and thus require the presence of a radical photoprimer and of said biocidal monomers and of said polymerisable compounds, photopolymerisable radically.

These said grafting primers are well known to the expert and are selected, especially, amongst the families of the following compounds:

1. Organic Peroxide Compounds, Especially:
peroxyesters, especially 1-dimethyl-3-hydroxybutyl peroxidecanoate, gamma-cumyl peroxidecanoate, gamma-cumyl peroxyheptanoate, t-amyl-peroxidecanoate, 2,5-dimethyl 2,5-di(2-ethylhexanoylperoxy)hexane, t-butylperoxypivalate, t-butylperoxy-2-ethylhexanoate, t-butylperoxyacetate, t-amylperoxyacetate, t-butylperbenzoate, t-amylperbenzoate;
hydroperoxides, especially tert-butyl hydroperoxides, amylhydroperoxide;
peroxyacetals, especially 1,1-di(t-butylperoxy)-cyclohexane, 1,1-di (t-butylperoxy)-3,3,5-trimethyl-cyclohexane, 1,1-di(t-amylperoxy)-cyclohexane, ethyl-3,3-di(t-butylperoxy)-butyrate; peroxidicarbonates such as di(n-propyl)peroxidicarbonates, di(sec-butyl) peroxidicarbonate and di(2-ethylhexyl)peroxidicarbonate;
diacylperoxides, especially benzoyl peroxide, urea peroxide, lauroyl peroxide, decanoyl peroxide;
2. inorganic peroxides, especially potassium persulfate, ammonium persulfate and hydrogen peroxide;
3. organic or inorganic peroxide compounds cited hereinabove, used in a mixture with:
either compounds selected amongst salts of Ag⁺, V²⁺, Ti²⁺, Co²⁺, Cu⁺, Fe²⁺, Ce²⁺, Na⁺and K⁺, and especially:
   nitrate, acetate, sulphate, carbonate, perchlorate of Ag⁺, V²⁺, Ti²⁺, Co²⁺, Ce²⁺, Cu⁺, Fe²⁺, or
   sulfite, hydrosulfite, bisulfite, metabisulfite, thiosulfate, sodium or potassium sulfide,
or reductive organic compounds, especially glucose, levulose, sorbose, hydrazine, hydroxylamine, amine, alcohol, tertiary diamine, mercaptan, organometallic compounds.
4. cerium Ce⁴⁺ or vanadium V⁵⁺ salts, that is, in the maximum oxidation state, especially salts of ammonium, nitrate or cerium or vanadium sulphate, which act such on substrates having hydroxyl or amine functions, by favouring the formation of active centres.
5. azo primers consisting of derivatives of azo compounds selected amongst diazoamino derivatives, diazothio derivatives, tetrazines, diazohydrates and diazoacetates, and more particularly: azo-bis-isobutyronitrile, azobiscumene, azo-bisiso-1,1,1-tricyclopropylmethane, 4-nitrophenyl-azo-triphenylemethane and phenyl-azo-triphenylmethane, this list not being exhaustive.

B. The Coupling Agents

The coupling agents act by creating chemical bonds between the substrate and the coating of said biocidal polymer.

These coupling agents can be employed in reactions photopolymerisation radically or cationically as a function of the reagent groups which they comprise. Nevertheless, they are more particularly employed where the utilisation of grafting primers radically is not possible or is difficult to carry out, especially as a function of the nature of the substrate, and more particularly again for substrates difficult to graft directly as substrates made of ceramic material, glass and/or metals.

The coupling agents are classed mainly in two distinct categories:
1. The coupling agents of silane type comprising (a) copolymerisable reagent groups with said biocidal monomers and said copolymerisable compounds, that is, radically or cationically, and (b) reagent groups allowing a covalent bond with groups of said substrate.

They can respond more particularly to the general formula (A):
R′ₙSiX′₍₄₋ₙ₎ (A)
in which:
R′ is an organic radical photopolymerisable radically or cationically, especially vinyl and methacryloyl groups (vinyltriethoxysilane, vinyltrimethoxysilane, 3-methacryloxypropyltrimethoxysilane, methacryloxidecyltriethoxysilane) for photopolymerisation radically or epoxy groups, (β-(3,4-epoxycyclohexyl)ethyl-trimethoxysilane, γ-glycidoxypropyltrimethoxysilane) and mercapto (3-mercaptopropyltrimethoxysilane) for photopolymerisation cationically, and
X′ is a hydroxyl group or another group easily hydrolysable, especially a methoxy, ethoxy or chloride group, so as to allow the chemical bond with the substrate.

These coupling agents of silane type are more particularly interesting for substrates comprising hydroxyl groups such as glass, ceramics but also certain materials based on polysaccharide or synthetic polymer.

2. Organometallic Coupling Agents:
such as titanates such as i-propoxy titanium tristearate, titanium tetrastearate, i-propoxy titanium trilaurate, isopropyl tri(dioctylphosphate) titanate, isopropyl tris(dodecyl benzene) sulfonyl titanate, neo-alkoxy tris [dioctylpyrophosphate]titanate,
phosphates such as (ethyl-), (butyl-), (hexyl-) (octyl-), (3,7-dimethyl-6-octenyl-), (2-(methacryloyoxy)isopropyl-), (6-(mercaptohexyl)-), (6-chlorohexyl-) phosphates,
zirconates such as i-propoxy zirconium tristearate, zirconium tetrastearate, i-propoxy zirconium trilaurate, neo-alkoxy tris [dodecyl benzene sulfonyl]zirconate,
chromates, aluminates, zirco-aluminates, cobalt salts, this list not being exhaustive.

The utilisation of grafting agents according to the present invention leads to a significant increase in the degree of grafting, UV radiation not being sufficient to form an adequate number of active surface centres. The grafting agent rate necessary to lead to efficacious fastening can vary between 0.01 and 10%.

Said copolymerisable compound must comprise reagent groups on the one hand allowing copolymerisation with said biocidal monomer, especially quaternary ammonium, and, on the other hand, covalent fixing on the substrate, owing to said grafting agents.

Said copolymerisable compounds have a single function only, for example acrylic, do not reticulate, they polymerise by giving linear chains and soluble copolymers, whereas bi or pluri functional compounds result in the formation of a three-dimensional reticulated and insoluble network of said grafted biocidal copolymer obtained.

The use of a reticulated copolymer according to the present invention, especially produces coating thicknesses which are more significant, as well as other advantages such as improved properties of resistance to chemical agents, improved mechanical characteristics, especially in terms of hardness and resistance to abrasion, improved behaviour under environmental conditions such as humidity, variation in temperature, resistance to thermal and photochemical degradation.

So preferably, according to the present invention, said formulation comprises at least one bi or pluri functional copolymerisable compound producing photopolymerisation and grafting of a said reticulated biocidal copolymer.

In an advantageous embodiment, said copolymerisable compound comprises a mono acrylate monomer or oligomer (n₁=1) or pluri (n₁=2 to 6) functional of formula (II) 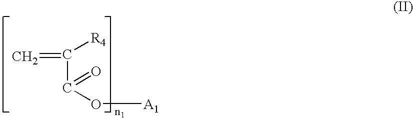
in which A₁ is an organic radical,
   R₄ is a hydrogen or a methyl, and
   n₁ is a whole number from 1 to 6.

More particularly, acrylate monomers or oligomers selected amongst the following compounds can be cited: methyl-acrylate, methylmethacrylate, ethylacrylate, iso-propylmethacrylate, n-hexylacrylate, stearylacrylate, allylacrylate, glycerol triacrylate, ethylene glycol diacrylate, diethylene glycoldiacrylate, triethylene glycol dimethacrylate, 1,3-propanediol diacrylate, 1,3-propanediol dimethacrylate, trimethylol propane triacrylate, 1,2,4-butanetriol trimethacrylate, 1,4-cyclohexanediol diacrylate, pentaerithrytol triacrylate, pentaerithrytol tetraacrylate, pentaerithrytol tetramethacrylate, sorbitol hexaacrylate bis [1-(2-acryloxy)]-p-ethoxyphenyledimethylemethane, bis[1-(3-acryloxy-2-hydroxy)]-p-propoxyphenylmethane, bis-acrylate and bismethacrylate of polyethylene glycol of 200-500 molar mass, copolymerisable mixtures of the monomers described hereinabove and of the following acrylate oligomers: polyetheracrylates modified in amine, polyurethane acrylate, polyester acrylate, polyether acrylate, acrylate multifunctional modified in amine, polyester hexaacrylate modified in fatty acid, polyester tetraacrylate, polyester methacrylate functionalised in acid, hexafunctional polyester acrylate, hexafunctional polyester acrylate modified in fatty acid, aliphatic urethane diacrylate, aliphatic urethane triacrylate, hexafunctional aliphatic urethane acrylate, silicone acrylate.

Preferably, according to the present invention, a formulation will comprise more particularly at least one at least bi functional compound of formula (II).

These said copolymerisable compounds of acrylate type of formula (II) require copolymerisation by photopolymerisation radically and thus require the presence of radical photoprimers in the formulation.

According to another embodiment of the process according to the present invention, said polymerisable compound comprises a mono epoxide monomer or oligomer (n₂=1), di (n₂=2) or tri (n₂=3) functional responding to the following general formula (III):

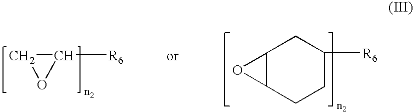

In which n₂ is a whole number from 1 to 3, and

R₅ is a radical of an organic radical.

More particularly, epoxides selected amongst the following compounds can be cited: 3,4-epoxycyclohexylmethyl-3,4-epoxycyclohexane carboxylate (Cyracure UVR 6105 and 6110 marketed by Union Carbide Corp.), 3,4-epoxy-6-methylcyclohexylmethyl-3,4-epoxy-6-methylcyclohexene carboxylate (ERL-4221), bis(3,4-epoxy-6-methylcyclohexylmethyl)adipate (Cyracure® UVR 6128 marketed by Union Carbide Corp.), octadecylene oxide, epichorhydrine, styrene oxide, vinylcyclohexene oxide, glycidol, glycidyl methacrylate, bisphenol A diglycidyl ether (EPON® 828, 825, 1004 and 1010 marketed by Shell Chemical Co), vinylcyclohexene dioxide (ERL-4206 marketed by Union Carbide Corp.), bis (2,3-epoxycyclopentyl ether) (ERL-0400 marketed by Union Carbide Corp), polypropylene glycol modified with epoxy (ERL 4050 and ERL-4052 marketed by Union Carbide Corp.), dipentene dioxide (ERL-4269), polybutadiene epoxide (Oxiron 2001 marketed by FMC Corp.), siliconised resin containing epoxy, flame-retarded resin epoxy (Dow Chemical Co.), 1,4-butanediol diglycidyl ether of phenolformaldehyde novolac (DEN-431 and DEN 438 marketed by Dow Chemical Co.), vinylcyclohexene monoxide 1,2-epoxyhexadecane (UVR-6216 marketed by Union Carbide Corp.), alkyl (C₈-C₁₂) glycidyl ethers (HELOXY Modifier 7 and 8, Shell Chemical Co.), 1,4-butanediol diglycidyl ether, neopentyl glycol diglycidyl ether, (HELOXY Modifier 68), cyclohexane dimethanol diglycidyl ether, trimethylol ethane triglycidyl ether, trimethylol propane triglycidyl ether, polyglycidyl ether of an aliphatic polyol, polyglycol diepoxide (HELOXY Modifier 67, 68, 107, 44, 48, 84 and respectively 32 marketed by Shell Chemical Co), bisphenol F diepoxides (EPN-1138 and GY-281 marketed by Ciba-Geigy Corp.), and glycidyl acrylates and methacrylates.

According to another embodiment, said copolymerisable compound comprises a vinyl ether monomer or oligomer responding to the following general formula (IV):
R₆—(O—CH═CH₂)_{1 or 2} (IV)
in which R₆ is a radical of an organic derivative.

More particularly, vinyl ethers selected amongst the following compounds can be cited: cyclohexanedimethanol divinylether, diethylaminoethylvinylether, tetraethyleneglycol divinylether, triethyleneglycol divinylether, cyclohexane dimethanol vinyl ether, cyclohexyl vinyl ether, n-dodecyl vinyl ether, lauryl vinyl ether, triethyleneglycol divinylether, 4-hydroxybutylvinylether.

Preferably, according to the present invention, said formulation comprises more particularly at least one at least bi functional copolymerisable compound of epoxide type of formula (III) or of vinyl ether type of formula (IV).

Said copolymerisable compounds of epoxide type of formula (III), or vinyl ether of formula (IV) hereinabove, require mechanisms of photopolymerisation cationically and thus the presence of cationic photoprimers.

In a variant embodiment, said photoprimer comprises a photoprimer radical comprising an organic compound containing at least a cycle phenyl substituted by a carbonyl, nitrogen or sulphur group.

More particularly, said photoprimer comprises a photoprimer radical comprising at least an organic compound containing chemical bonds in the molecule capable of being broken homolytically under UV radiation, and at least a phenyl cycle substituted by a carbonyl, phosphorous, nitrogen or sulphur group.

More particularly still, radical photoprimers selected amongst the following compounds can be cited:

1-hydroxy-cyclohexyl-phenyl-ketone, benzophenone, 2-hydroxy-2-methyl-1-phenyl-1-propanone, methylbenzoylformate, α,α-dimethoxy-α-phenylacetophenone, 2-benzyl-2-(dimethylamino)-1-[4-(4-morpholinyl)phenyl]-1-butanone, 2-methyl-1-[4-(methylthio)phenyl]-2-(4-morpholinyl)-1-propanone, diphenyl (2,4,6-trimethylbenzoyl)-phosphine oxide, phosphine oxide, phenyl bis (2,4,6-trimethyl benzoyl)-phosphine oxide, phosphine oxide, phenyl bis(2,4,6-trimethyl benzoyl).

The compounds hereinabove are marketed by the company Ciba Specialty Chemicals Inc. under the following references: Irgacure® 184, 500, 1000, 2959, 651, 369, 907, 1300, 819, 819DW, 2005, 2010, 2020, Darocur® 1173, MBF, TPO, and 4265.

In another variant embodiment, the said photoprimer comprises a cationic photoprimer comprising ionic compounds containing organic cations such as aryl sulfonium or aryl iodonium compounds with counter-ions such as SbF₆⁻, PF₆⁻, AsF₆⁻, BF₄⁻, PO₄⁻ capable of attacking electrophillically said biocidal monomer or said copolymerisable compound, by creating cationic species subsequently capable of continuing polymerisation.

More particularly, said cationic photoprimer is an aryl sulfonium salt, especially triaryl sulfonium phosphate, triarylsulfonium antimonate, triarylsulfonium hexafluorophosphate, (UVI 6974, UVI 6992), or an aryl iodonium salt such as diaryliodonium hexafluoroantimonate, bisdodecylphenyliodonium hexafluoroantimonate, iodonium, (4-methylphenyl)[4-(2-methylpropyl)phenyl]-hexafluorophosphate (1-) (CGI 552) marketed by Ciba® Specialty Chemicals or by Union Carbide Corporation.

The concentrations of the compounds of the formulation utilised can vary within fairly substantial limits as a function of the physical-chemical, mechanical and bacteriological properties to be obtained.

In a preferred embodiment, said formulation comprises different constituents in the following proportions by weight for a total of 100%, namely:
1) 5 to 95%, preferably 5 to 50%, of said biocidal monomers,
2) 5 to 95%, preferably 10 to 75%, of said copolymerisable compounds,
3) 1 to 10% of said photoprimers, and
4) 0.01 to 10% of said grafting agents.

According to another subsidiary characteristic of the present invention, said formulation comprises additive constituents selected amongst:
a compound having hydroxyl functions,
another polymerisable compound of anhydride type or derivatives, of styrene type or its derivatives or of cyanoacrylate type,
an additive selected amongst softening, stabilising, dispersing, flame retarding, dyeing, plastifying agents, touch improver, adhesion agents.
solvents, reactive or not, utilised especially for decreasing viscosity.

These constituents or additives are well known to the expert.

More particularly, the following additive constituents are cited:
As solvent, reactive or not, one of the acrylate or methacrylate monomers responding to the general formula (II), alcohols, water or other solvents.

As constituents having hydroxyl functions, alcohols, monoalkyl ethers of polyoxyalkylene glycols, monoalkyl ethers of alkylene glycols, 1,2-ethanediol, 1,3-propanediols, 1,4-butanediol, 1,6-hexanediol, 1,8-octanediol, 2-ethyle-1,6-hexanediol, bis (hydroxymethyl) cyclohexane, 1,18-dihydroxyoctadecane, 3-chloro-1,2-propanediol, polyhydroxyalkanes (glycerine, trimethylolethane, pentaerithritol, sorbitol) and polymers containing hydroxyls such as polyoxyethylene and polyoxypropylene di- or triols, polytetrahydrofurane, copolymers of hydroxypropyl and hydroxyethyl acrylates and methacrylates and other radically polymerisable monomers, copolymers containing counterpart hydroxyl groups formed by hydrolysis, polyvinyl acetal resins with OH counterparts, modified cellulosic polymers, polyesters, polylactones, polycaprolactones, polyalkadienes having a hydroxyl group at the end of the chain,
As other polymerisable compounds, cyanoacrylate adhesives: diethyl 3,3′-(1,4-phenylene)bis(2-cyanoacrylate), ethyl 3-(3-chloro-4-methoxyphenyl)-2-cyanoacrylate, ethyl 2-cyanoacrylate, 3-(5-(2-chloro-5-(trifluoromethyl)phenyl)-2-furyl)-2-ethyl cyanoacrylate, 3-(5-(2-chlorophenyl)-2-furyl)-2-ethyl cyanoacrylate, 3-(5-(3-chlorophenyl)-2-furyl)-2-ethyl cyanoacrylate, 3-(5-(4-chlorophenyl)-2-furyl)-2-ethyl cyanoacrylate, 3-(5-bromo-2-furyl)-2-cyanoacrylate, 3-(5-(4-(aminosulfonyl)phenyl)-2-furyl)-2-cyanoacrylate; anhydrides: 2,3-dibromomaleic anhydride, maleic anhydride, 2-ethyl-3-propylacrylic anhydride; styrene derivatives: styrene, α-methylstyrene, divinyl benzene.

In a variant embodiment of the process according to the present invention, said formulation comprises:
at least one grafting primer preferably comprising an organic peroxide compound or a cerium salt Ce⁴⁺, and
at least one said radical photoprimer.

According to another variant embodiment of the process according to the present invention, said formulation comprises:
at least one said cationic or radical photoprimer, and
at least one said coupling agent of silane type.

This second claim variant is more particularly appropriate for grafting on substrates comprising hydroxyl functions.

The object of the present invention likewise is a solid substrate comprising a polymer exhibiting biocidal properties, grafted to its surface, obtained by the process according to the present invention.

In an embodiment, said solid substrate is constituted by a natural or synthetic organic material, preferably a material of plastic type, a material based on natural polymer such as polysaccharides.

More particularly still, said substrate is selected amongst fibrous textile or non-woven organic materials, based on synthetic or natural threads or fibres.

In another embodiment, said solid substrate is constituted by an inorganic material, preferably a ceramic material or glass or even metal.

The grafting agents can be selected as a function of the type of substrate:
For substrates having hydroxyl functions (glass, cellulose, wood), said grafting primers such as metallic salts can be used as said grafting agents, especially cerium salts if the formulation contains a radical photoprimer, or of said coupling agents if the formulation comprises cationic photoprimers and constituents implying cationic photopolymerisation, especially coupling agents such as compounds of silane type.
For grafting on polyester, polyurethane, cellophane, polyethylene and polypropylene substrates, a grafting primer such as silver nitrate/urea peroxide couple or ammonium persulfate can be used.
For hydrophilic polymers such as poly(vinyl alcohol), poly(hydroxyethylmethacrylate), poly(acid acrylic), poly(vinylpyrrolidone), poly(alkylene glycol) and gelatine, grafting primers such as peroxides, persulfates, redox oxidising/reductive couples or coupling agents such as compounds of silane type can be used.
For substrates such as ethylene vinyl acetate copolymers, ethylene ethyl acrylate copolymers, benzoyl peroxide, tert-butylhydroperoxide, methyl ethyl ketone peroxide and ferrous ammonium sulfate can be used as grafting primers. As a general rule, grafting agents must be slightly soluble in the photopolymerisable formulation, and at the same time they must have good affinity for the substrates being used in order to favour grafting and decrease the speed of the homopolymerisation process.
For composite substrates comprising materials of different nature such as gel-coat based on copolymer polyester/styrene resin loaded with silica, the two types of grafting agents, namely a coupling agent and a grafting primer can be used advantageously conjointly.

The type of grafting agents also depends on the formulation. If an aqueous formulation is used, hydrosoluble grafting agents will be used and, if non-aqueous formulations are used, it is preferred to use peroxides or soluble redox couples in the organic products. In all cases, the grafting agents must have good compatibility with the substrate to be grafted.

Other characteristics and advantages of the present invention will emerge from the detailed embodiment examples which follow hereinbelow.

### EXAMPLE 1

### Synthesis of Biocidal Monomers

1.1 Synthesis of Bromide Methacryloylethyl Dimethyloctyl Ammonium

4.71 g (0.03 moles) of dimethylaminoethylmethacrylate and 5.79 g (0.03 moles) of octyl bromide are added to 10 ml of ethanol. The solution is then agitated in an oil bath, at 60° C., for 48 hours. The dosage of Br⁻ ions proves that after this reaction time the conversion achieved is 99%. This mixture is then cooled to ambient temperature and precipitated in ethyl ether. The resulting precipitate is then filtered and washed several times in ether.

The reaction diagram is the following:

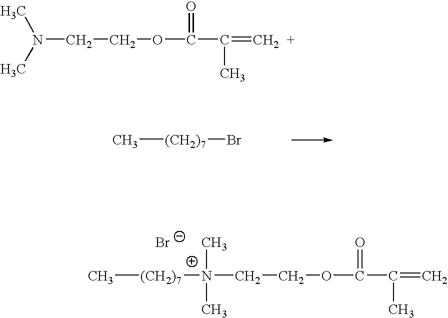

1.2 Synthesis of Methacryloylethyldimethyoctylammonium Iodide.

4.71 g (0.03 moles) of dimethylaminoethylmethacrylate and 7.2 g (0.03 moles) of octyl iodide are added to 10 ml of ethanol. The solution is then stirred in an oil bath, at 60° C., for 48 hours. The dosage of the I⁻ ions proves that after this reaction time the resulting conversion is 99.1%. This mixture is then cooled to ambient temperature and precipitated in ethylic ether. The precipitate obtained is then filtered and washed several times with ether.

1.3 Quaternisation of Dimetylaminopropylmethacrylamide by Decyl Bromide.

4.68 g of dimethylaminopropylmethacrylamide and 6.63 g of decyl bromide are dissolved in 10-15 ml of ethanol and the mixture is maintained for 72 hours at 60° C. under strong agitation. The dosage of the Br⁻ ions is used to determine the reaction yield which is around 98%, considered as satisfactory. The solvent is then removed by means of a rotavapor and the quaternary salt obtained is a yellowish viscous liquid which can be utilised as such in the formulations.

In respecting the same work method, quaternary salts were also synthesised by using bromides and octyl, decyl, dodecyl, tetradecyl and hexadecyl iodides.

1.4 Synthesis of Other Dimethylaminoethylmethacrylate Quaternary Salts. Change of Counter Ions.

0.5 mole of sodium salicylate, dissolved also in 0.5 l isopropylic alcohol, is added to a solution of 0.5 mole of methacryloylethyldimethyhexadecylammonium bromide in 0.5 l isopropylic alcohol. The second solution is introduced to the first drop by drop. The mixture is stirred so that the temperature is raised to 60° C. The temperature is kept constant for 8 hours. The mixture obtained is then cooled to ambient temperature, then filtered. Two thirds of the solvent is removed by distillation at reduced pressure and an equal quantity of water is added. The salt is then crystallised in an ice bath, then filtered.

In the same way other quaternary ammonium salts with divers counter ions, such as benzoate, acetate, undecylenate, acetyl or salicylate can be synthesised. The solvent can be replaced by another polar solvent or a mixture of solvents, as a function of the organic salt utilised for quaternisation (water/alcohol, acetone/benzene, chloroform/benzene mixture).

1.5 Quaternisation of 2[2-(dimethylamino)ethoxy]ethanol by Dodecyl Bromide.

54 g of 2[2-(dimethylamino)ethoxy]ethanol and 99, 7 g of dodecyl bromide are introduced to a 250 ml bicolour balloon equipped with a refrigerant. The solution is homogenised by means of a magnetic agitator, in an oil bath. It is then heated at 64° C., for 21 hours. The resulting conversion is 99%. As it cools, a solid product of a slightly yellowish colour is obtained.

1.6 Quaternisation of Didecylmethylamine by 3-chloro-1,2-propanediol.

1 mole of didecylmethylamine reacts with 1 mole of 3-chloro-1,2-propanediol in nitromethane in reflux, with stirring for 60 hours. The solvent is then eliminated by using the rotavapor, under vacuum. The quaternary salt is in the form of a highly viscous yellow-brown residue.

1.7 Quaternisation of Trioctylphosphine by Methylstyrene Chloride

The reaction was performed en masse. 4.31 g (0.028 moles) of methylstyrene chloride are added to 10.49 g of trioctylphosphine (0.028 moles). The mixture is stirred for 5 hours at 50° C., using a magnetic agitator. The quaternary salt starts to form after an hour, and presents as a yellow precipitate.

1.8 Quaternisation of Trioctylphosphine by 3-chloro-1,2-propanediol.

The reaction was performed en masse. 2.22 g (0.02 moles) of 3-chloro-1,2-propanediol was added to 7.4 g of trioctylphosphine (0.02 moles). The mixture is stirred for 92 hours at 130° C., using a magnetic agitator. The biphasic system becomes homogeneous and coulometric analysis reveals a quaternisation yield of 96.4%. The quaternary salt thus formed presents as a clear viscous liquid.

### EXAMPLE 2

### Photopolymerisation and Grafting on a Substrate

To perform photopolymerisation of the formulations described hereinbelow on a laboratory scale Novacure® N 2001-A1 apparatus by EFOS was used, containing a 100 W mercury lamp and a filter enabling elimination of IR radiation and irradiation of the sample at a wavelength of 360-500 nm. The apparatus is fitted with a dual-head light guide having a diameter of 3 mm. In order to follow the photopolymerisation process in real time, measure the reaction enthalpy and determine the induction time, the Novacure® apparatus can be coupled to a DSC Pyris® 1 marketed by Perkin Elmer. For photopolymerisation performed on samples of more substantial dimensions Fusion UVF-300 equipment with a conveyor was used.

2.1 Grafting on Cotton-Based Fabrics or Polyester/Cotton Mixture

The grafting of biocidal monomers was carried out in two different ways.

2.1.1. Treatment in One Step

A solution is prepared containing 15% unsaturated biocidal monomer, 5% of said copolymerisable compound (for example polyethylene glycol diacrylate), 0.5% grafting primer Ce(NO₃)₆(NH₄)₂ (ammoniacal cerium nitrate), and 5% radical photoprimer Irgacure® DW819 (bis(2,4,6-trimethylbenzoyl)-phenylphosphine oxide), 40% demineralised water and 34.5% ethanol. A 2×2 cm sample of fabric is soaked in 0.5 g of this solution. After impregnation it is irradiated for 10 seconds on each side at a luminous intensity of 200 mW/cm² with a UV lamp emitting in the 280-500 nm range and dried for 10 minutes in a drying cabinet at 100° C. The method can be applied on an industrial scale by impregnating the textile by the padding method. As a function of the cotton content of the fabric, the quantity of formulation absorbed is between 80-180 g/cm² (80 g/cm² for the PE/cotton mixtures and 180 g/cm² for the pure cotton). The textile then passes between two UV sources at an intensity of 100 to 1000 mW/cm², varying as a function of the reactivity of the constituents, emitting in the 280-500 nm range, at a speed of 10-40 m/min and dried in an oven tunnel at temperatures between 100-180° C. The reaction starts with UV decomposition of the photoprimer, followed by photopolymerisation and is completed thermally during passage through the oven tunnel. 2.1.2. Treatment in Two Steps

On a laboratory scale a sample of 2×2 cm fabric is soaked in 0.5 g of solution comprising 5% radical photoprimer Irgacure® DW819 (bis(2,4,6-trimethylbenzoyl)-phenylphosphine oxide) and 0.5% grafting primer Ce(NO₃) 6 (NH₄)₂ in water. It is then dried in a drying cabinet for 10 minutes at 100° C., then soaked in a second solution containing 15% unsaturated biocidal monomer, 5% polyethyleneglycoldiacrylate, 45% water and 35% ethanol. It is then irradiated for 10 seconds on each side at a luminous intensity of 200 mW/cm² with a UV lamp emitting in the 280-500 nm range and then dried in a drying cabinet at 100° C.

On an industrial scale the textile is first impregnated with a solution containing the Darocur® DW819 (bis(2,4,6-trimethylbenzoyl)-phenylphosphine oxide) photoprimer and the grafting primer Ce(NO₃)₆(NH₄)₂ in water, and dried at 100-180° C. Next the dry fabric containing photoprimer is passed to the second solution containing the biocidal monomer and optionally other compounds. As a function of the cotton content of the fabric, the quantity of formulation absorbed is between 80-180 g/cm². The textile is then irradiated between two UV sources with an intensity of 100 to 1000 mW/cm²—varying as a function of the reactivity of the constituents and emitting in the 360-500 nm range, at a speed of 10 to 40-m/min and dried in an oven tunnel at temperatures between 100-180° C.

The formulation utilised for treating the fabrics, promptly, can contain other adjuvants such as:
softeners such as an emulsion of functional polysiloxane,
finishes such as a dispersion of vinyl polyacetate,
flame-retarding agents such as an emulsion of fluorocarbonated resin, and
touch and volume improvers such as an emulsion of acrylic copolymers.

2.1.3. Efficacy of the Grafting

After washing by ethanol (to remove the homopolymer) for 1 hour, at 60° C., analysis by retrodiffuse X rays coupled to electronic scanning microscopy, carried out on a fibre reveals a content of 6.57% by weight of bromine. The presence of bromide ions (counter ions) indirectly proves the presence of surface ammonium quaternary cations.

To prove the efficacy of the grafting by the bactericidal monomer, a sample of cotton fabric (80%)/polyester (20%) is soaked in the second treatment step with a solution containing just the ammonium quaternary monomer, without any reticulation agent. A treatment example (1) carried out according to this principle is as follows: a 2×2 cm sample of textile is soaked in a first step in 0.5 g of solution comprising 5% of bis(2,4,6-trimethylbenzoyl)-phenylphosphine oxide (Irgacure DW819), 0.5% of Ce(NO₃)₆(NH₄)₂ and 94.5% of water. After this period the sample is dried at 100° C., then soaked in a second solution containing 20% ammonium quaternary monomer in water. After UV radiation, it is dried at 100° C., for 10 minutes and washed in ethanol for 1 hour, at 60° C., to remove the homopolymer which has formed. The existence of the surface-grafted homopolymer is proven, since analysis performed by X rays reveals a large percentage of bromide and thus of quaternary ammonium of 3.75%. In another example (2) where the formulation comprises no grafting primer, the measured rate of bromine ions is only 0.3% by weight.

More evidence of grafting by the bactericidal monomer is contributed by IRTF spectroscopy (Spectrum One by Perkin Elmer®), in ATR mode (Attenuated Total Reflectance). In example 1) after washing in ethanol a band is observed which is characteristic of the methylene groups at 2950-2850 cm⁻¹ attributed to the R₃ group borne by the quaternary ammonium of the grafted homopolymer, much more intense than in example 2.

More evidence contributed to the efficacy of the grafting of the fabric is obtained by analysing the samples by thermogravimetric analysis, under nitrogen, using Pyris 1 ATG (Perkin Elmer®) equipment. The thermal program is the following: isothermal for 1 minute at 40° C., followed by heating from 40° C. to 500° C. at a speed of 20° C./min.

The software for interpreting the data helps determine temperature values for start of thermal decomposition of the substrate To for each of the untreated compounds of the fabric (441° C. for the polyester and 380° C. for the cotton). After grafting of the cotton with the bactericidal formulation by the two-step process, followed by washing, displacement of the peak corresponding to the cotton towards lower temperatures is observed (value Tₒ equal to 332° C., less than that of the unmodified cotton), a fact which constitutes proof of the chemical modification of the latter. On the contrary, the treatment is carried out under the same conditions, but without grafting primer and almost ineffective, since the temperature Tₒ is very close to that of the cotton or 375° C.

Another test comprises determining the bromide content for a sample of bactericidal treated fabric (cotton/polyester 80%/20%) in a single step. A piece of 2×2 cm fabric is soaked in 0.5 g of solution containing 20% methacryloylethyldimethyltetradecyl ammonium bromide, 0.5% ammoniacal cerium nitrate and 5% bis(2,4,6-trimethylbenzoyl)-phenylphosphine oxide (Irgacure DW819) in 74.5% water. After UV radiation for 10 seconds on each side, the sample is dried at 100° C. for 5 minutes, then washed in ethanol for 1 hour, at 60° C. X ray analysis of the sample reveals low mass content of bromine ions, around 0.6%. This test proves that treatment of the textile carried out in two steps is much more efficacious than that done in a single step.

There again, IRTF spectroscopy confirms the significant rate of grafting in the case of the two-step process.

ATG analysis done in the case of the sample treated in a single step results in a temperature To equal to 343° C., whereas for the sample grafted in two steps this value is lower, namely 332° C. When the rate of grafting increases, this temperature Tₒ drops.

The general activation mechanism of the cellulose by the cerium salts is described hereinbelow:

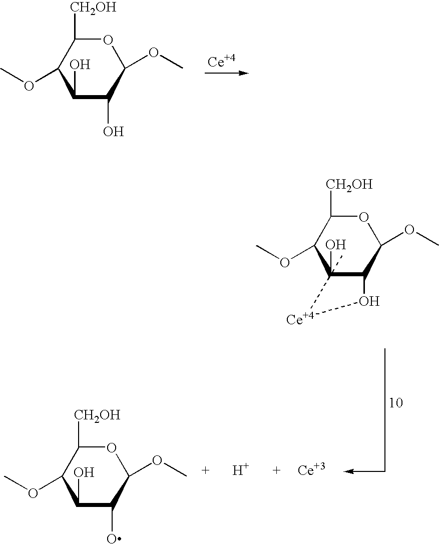

The active centres formed in the surface constitute the sites where the grafts obtained from methacryloylethyldimethyltetradecyl ammonium bromide and polyethyleneglycoldiacrylate are formed, for example:

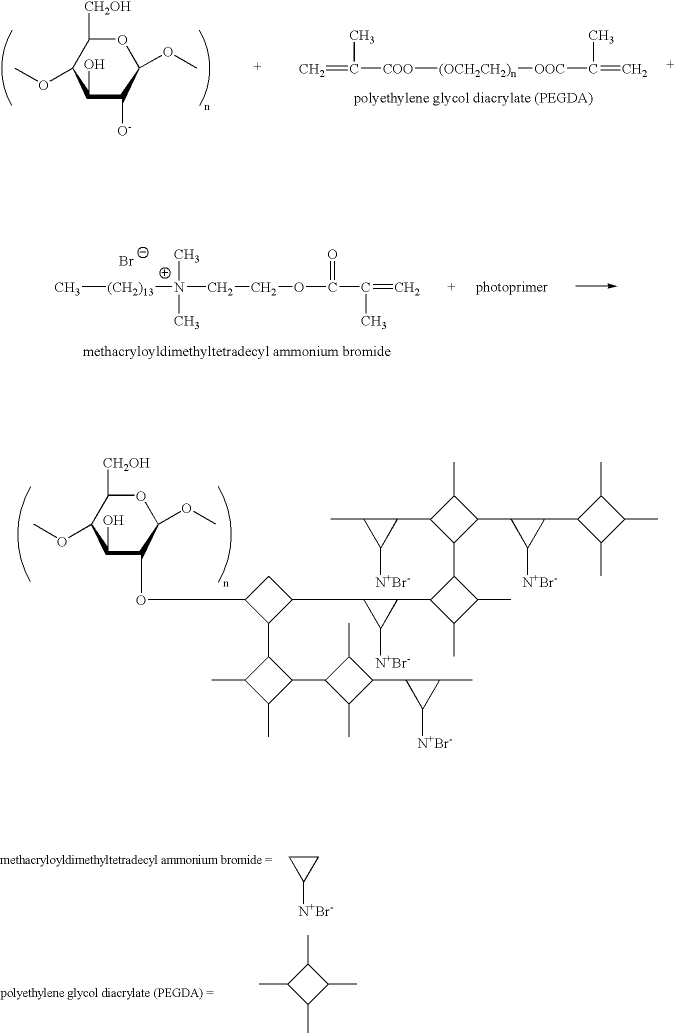

2.2 Treatment and Grafting on a PVC Plate

A fine layer of photopolymerisable mixture compound is deposited on a 2×2 cm square of PVC, said mixture comprising 20% methacryloylethyldimethylhexadecyl ammonium tetrafluoroborate, 41% 3,4-epoxycyclohexylmethyl-3,4-epoxycyclohexane carboxylate (Cyracure® UVR 6105) such as said copolymerisable compound, 5% triarylsulfonium antimonate (Cyracure® UVI 6974) as cationic photoprimer, 10% 1,4-butanediol as solvent of the bactericidal compound, 20% tetrapropyleneglycoldiacrylate such as other said copolymerisable compound, 3% Irgacure® 2020 (mixture of 80% 1-hydroxy-cyclohexyl-phenyl-ketone and 20% phenyl bis (2,4,6-trimethyl benzoyl)-phosphine oxide) as radical photoprimer, and a grafting primer in the form of a redox couple formed by 0.5% benzoyl peroxide and 0.5% dimethylphenylamine as reductive organic compound. This is radiated for 20 seconds at an intensity of 1000 mW/cm². The coating obtained is bound chemically to the substrate, because of covalent bonds which are formed on the surface, as witness the X ray test.

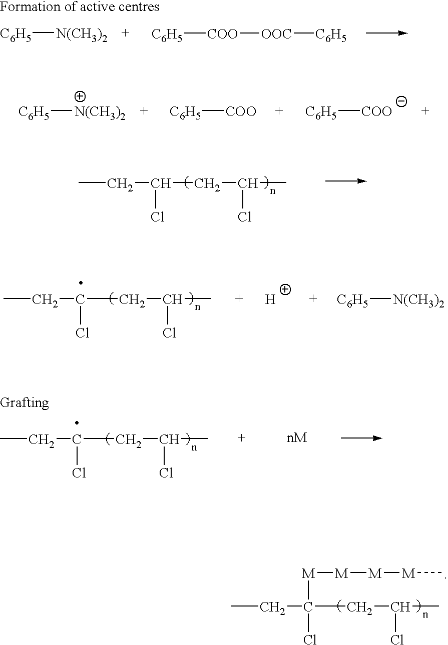

The priming and grafting mechanism is presented hereinabove:

M represents the bactericidal monomers or said copolymerisable acrylate compounds.

Furthermore, the cationic photoprimer favours the polymerisation of said copolymerisable compound of epoxide type.

2.3. Grafting on Ceramic Plates.

Photopolymerisation is carried out in this case by a hybrid radical/cationic mechanism. The formulation comprises 45% bis(3,4-epoxy-6-methylcyclohexylmethyl) adipate (Cyracure UVR 6128) as said copolymerisable compound, 20% methacryloylethyldimethyltetradecyl ammonium tetrafluoroborate, 2% triarylsulfonium antimonate (Cyracure UVI 6974) as cationic photoprimer, 30% polyethylene glycol dimethacrylate as other said copolymerisable compound, 2% diphenyl (2,4,6-trimethylbenzoyl)-phosphine oxide (Darocur TPO) as radical photoprimer and 1% vinyltrimethoxysilane as coupling agent. 2 g of this mixture are deposited on a surface of 100 cm² and irradiated for 20 seconds at an intensity of 500 mW/cm².

The silane coupling agent reacts on the one hand on the surface hydroxyl groups of the ceramic substrate by means of methoxy groups by creating bonds of ether type according to the following reaction diagram

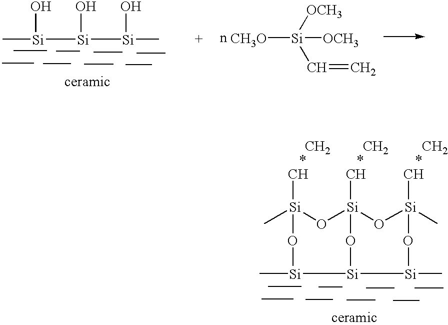

On the other hand, the vinyl groups of silane then participate radically in the photoprimed copolymerisation by reacting with the biocidal monomer and with said copolymerisable acrylate compound according to the following reaction diagram:

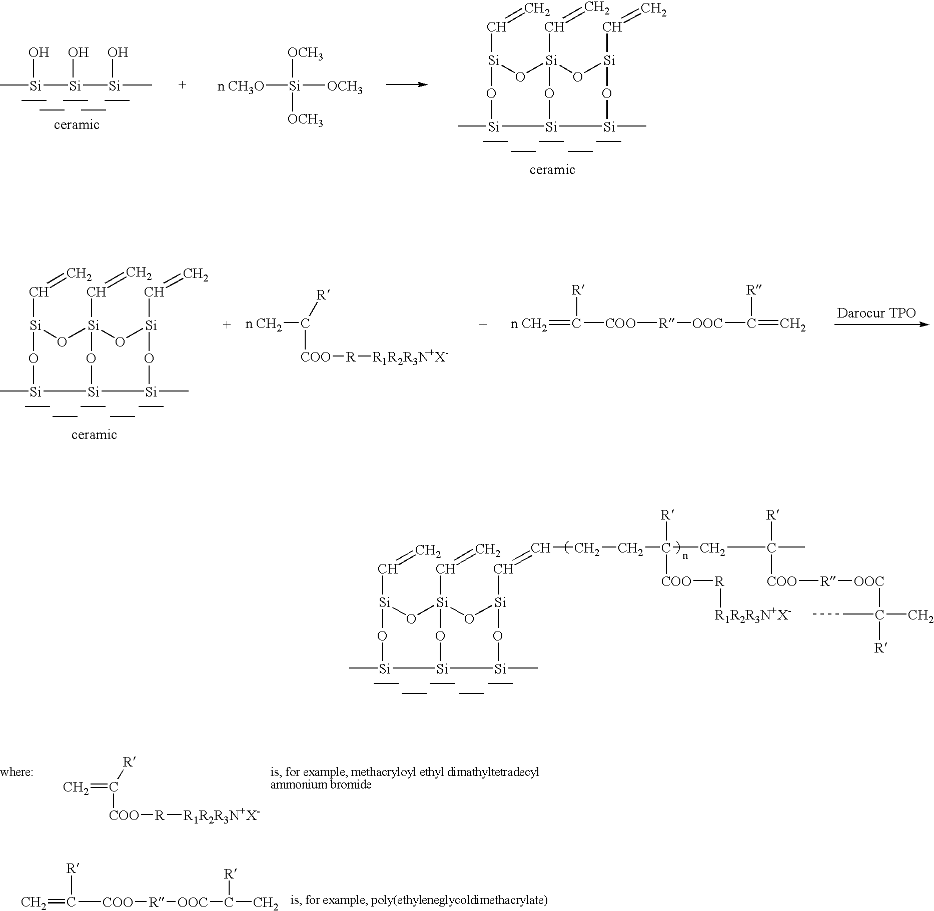

The cationic photoprimer benefits copolymerisation of the copolymerisable epoxide compound.

Grafting on porcelain squares

The upper layer of the porcelain plates utilised here has an inorganic chemical structure having the following composition: SiO₂ 55.3%, Al₂O₃ 8.3%, MgO 2.1%, K₂O 3.8%, CaO 8.5%, ZnO 11.9%, ZrO₂ 7.4%. It therefore contains a significant percentage of silica.

The surface des porcelain squares can be treated similarly to the following process:

1 g of formulation is deposited onto a porcelain square of 25 cm², comprising 34% 3,4-epoxycyclohexylmethyl-3,4-epoxycyclohexane carboxylate (Cyracure UVR 6105), 1% triarylsulfonium antimonate (Cyracure UVI 6974) as cationic primer, 42% tetraethyleneglycoldiacrylate, 10% methacryloylpropyl dimethylhexadecyl ammonium tetrafluoroborate, 3% radical photoprimer (2,4,6-trimethylbenzoyl)-phosphine oxide (Darocur TPO) and 10% 3-(trimethoxysilyl)propylmethacrylate as coupling agent. This is irradiated via UV for 20 seconds at 500 mW/cm². The film of bactericidal polymer is grafted onto the surface of the porcelain square since it does not detach even after washing.

2.4. Bactericidal Treatment and Grafting Performed on Wood.

The treatment of bois by UV technique can be performed either by a radical mechanism, or by a cationic mechanism.

2.4.1. By way of example, the base formulation can contain: 50% polyurethane acrylate (Laromer UA 19 T by BASF) as said copolymerisable compound, 25% tripropyleneglycoldiacrylate as other said copolymerisable compound, 19% methacryloylethyldimethyldodecycl ammonium bromide, 5% Irgacure® 2020 by Ciba Geigy as radical photoprimer and as grafting primer 1% 3-(trimethoxysilyl)propylmethacrylate.

2.4.2. By way of example, 1.5 g of base formulation is applied to a wood plate (2×2 cm) containing: 10% dimethyloctylethoxyethanolammonium tetrafluoroborate, 70% 3,4-epoxycyclohexylmethyl-3,4-epoxycyclohexane carboxylate (Cyracure UVR 6105) as said copolymerisable compound, 5% triarylsulfonium hexafluorophosphate (Cyracure UVI 6974) as cationic photoprimer, 10% 1,4-butanediol as reagent solvent of the bactericidal compound and 5% glycidoxypropyltrimethoxysilane as coupling agent. This is irradiated for 20 seconds at an intensity of 1000 mW/cm².

The rôle of the silane coupling agent is to augment the grafting on the cellulosic substrate, by creating an interface according to the following reaction diagram:

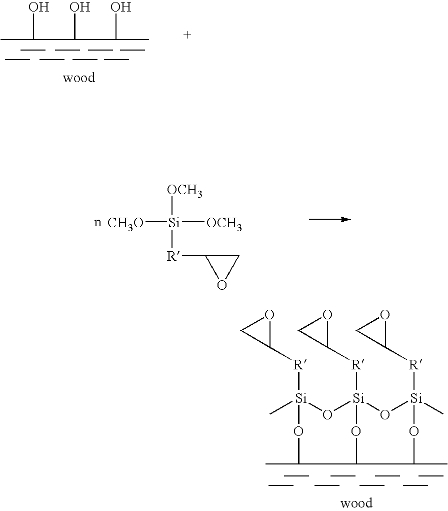

The epoxy groups of the silane coupling agent then copolymerise with the epoxides and the cationic bactericidal monomer of the formulation by thus producing chemical grafting of the bactericidal coating.

Priming of the polymerisation of the copolymerisable epoxide compounds is done according to the following reaction diagram:

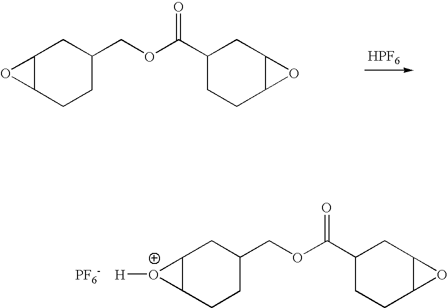

And, copolymerisation and grafting on the interface constituted by the coupling agent are carried out according to the following reaction diagram:

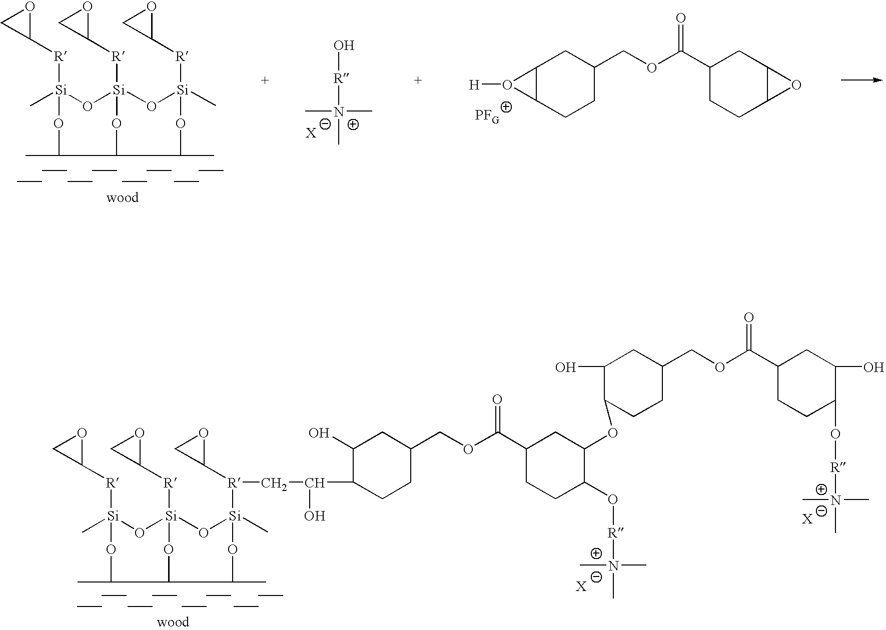

As a function of the desired properties and of the type of application of the formulation, the type of epoxide and the ratios of the constituents of the formulation can be varied.

2.5. Bactericidal Treatment and Grafting on Glass

0.5 g of photosensitive formulation containing: 3% 3-(trimethoxysilyl)propylmethacrylate, 5% radical photoprimer (2,4,6-trimethylbenzoyl)-phosphine oxide (Darocur TPO), 10% methacryloylethyldimethyldodecyl ammonium bromide and 82% epoxy acrylate (Laromer 8986 by BASF) are applied to a plate of glass. This is irradiated for 10 seconds at an intensity of 500 mW/cm². The superficial layer formed exhibits good adherence to the support because of the covalent bonds which are formed between the bactericidal monomer, the polymerisable compound and the silanols present on the surface of the glass plate, by means of the coupling agent (3-(trimethoxysilyl) propyl methacrylate). The action mechanism is similar to those presented for the treatment of the plates of wood and ceramic.

2.6. Bactericidal Treatment and Grafting on Gel-Coat

0.2 g of a photosensitive formulation containing: 10% methacryloylethyldimethyloctyl ammonium bromide, 82% polyethylene glycol diacrylate, 2% 3-(trimethoxysilyl)propylmethacrylate, 4% radical photoprimer (2,4,6-trimethylbenzoyl)-phosphine oxide (Darocur TPO), and a redox couple acting in organic medium, formed from 0.5% cobalt octoate and 1.5% methylethyl ketone peroxide are applied to a 5×5 cm plate of “gel-coat” (polyester resin of isophtalic type charged with silica, reticulated with styrene). This is irradiated for 20 seconds at an intensity of 1000 mW/cm². Likewise in this case, the superficial layer formed exhibits good adherence to the support due to covalent bonds which are formed between the bactericidal monomer, the polymerisable compound and the surface of the gel-coat plate, by means of active centres which are formed under the action of redox grafting primers. The action mechanism is similar to that presented for treatment of the PVC plates. For its part the coupling agent acts on the surface hydroxyl, belonging to the mineral or existing charges at the end of the polyester chain.

### EXAMPLE 3

### Results of the Bactericidal Tests on Textiles Grafted with Bactericidal Monomers by UV as per Example 2

Two Series of Samples were Tested:
1. textile (cotton/polyester mixture)
   1.1 before treatment (reference)
   1.2 after treatment of example 2.1
   1.3 after treatment and washing
2. glass plate
   2.1 no treatment (reference)
   2.2 after treatment of example 2.5.
   2.3 after treatment and washing

The treatment consisted of soaking (for the textile) or deposit on the glass plate of a formulation comprising inter alia an antigerm monomer and a photoprimer and a grafting agent, followed by UV radiation.

The prepared samples were washed in the same following manner: the cotton fabrics were washed with digestive water for 30 h, at 60° C., under strong agitation; the glass plates were immersed in hot water for 4 hours. All the samples were rinsed with distilled water and dried. For each of them, including an untreated test fabric, biocidal efficacy was verified on two different stubs: Staphylococcus Aureus (bacteria) and Aspergillus Niger (fungi).

Bactericidal efficacy by diffusion and by contact was verified. The process consists of placing the thus grafted fabric and a bacterial suspension of the abovementioned stubs in contact for determined time. In a first period, efficacy by diffusion (*) was determined by measuring an inhibition zone around samples deposited over 24 hours on the surface of a pre-contaminated gelose medium. Next, after 24 hours, efficacy by contact (**) of the preceding samples was determined by numbering, after deposit of the inoculum according to a procedure adapted to French Standard XP G 39-010.

Each determination (inhibition zone and numbering) was carried out on three samples (values given as . . . / . . . / . . . ) the value appearing between parentheses, being an average value of the latter.

Test fabric (1.1), treated (1.2) and treated then washed (1.3) on a Staphylococcus aureus (bacteria) stub.

| | | Treated and | Treated and |
|---|---|---|---|
| | Tests | not washed | washed |
| | (1.1) | (1.2) | (1.3) |
| 24 contact | 0/0/0 | 2/3/2 | 2/2/1 |
| hours* | (0) | (2.3) | (1.7) |
| Numbering | >10⁶/>10⁶/ | 0/0/0 | 0/0/0 |
| after 48 | >10⁶ | (0) | (0) |
| hours** | (>10⁶) | | |

Test fabric (1.1), treated (1.2) and treated then washed (1.3) on Aspergillus Niger (fungi) stub.

| | | Treated and | Treated and |
|---|---|---|---|
| | Tests | not washed | washed |
| | (1.1) | (1.2) | (1.3) |
| 24 contact | 0/0/0 | 2/2/2 | 0/0/0 |
| hours* | (0) | (2) | (0) |
| Numbering | >10⁶/ | 0/0/0 | 100/190/130 |
| after 48 | >10⁶/>10⁶ | (0) | (140) |
| hours** | (>10⁶) | | |

Glass test plate (2.1), treated (2.2) and treated then washed (2.3) on Staphylococcus aureus (bacteria) stub.

| | | Treated and | Treated and |
|---|---|---|---|
| | Tests | not washed | washed |
| | (2.1) | (2.2) | (2.3) |
| 24 contact | 0/0/0 | 5/4/5 | 5/4/4 |
| hours* | (0) | (4.7) | (4.3) |
| Numbering | >10⁶/ | 0/0/0 | 0/0/0 |
| after 48 | >10⁶/>10⁶ | (0) | (0) |
| hours** | (>10⁶) | | |

Test glass plate (2.1), treated (2.2) and treated then washed (2.3) on Aspergillus Niger (fungi)

| | | Treated and | Treated and |
|---|---|---|---|
| | Tests | not washed | washed |
| | (2.1) | (2.2) | (2.3) |
| 24 contact | 0/0/0 | 6/4/4 | 5/5/4 |
| hours* | (0) | (4.7) | (4.7) |
| Numbering | >10⁶/>10⁶/ | 160/200/190 | 340/270/230 |
| after 48 | >10⁶ | (183.3) | (280) |
| hours** | (>10⁶) | | |

### Conclusions

Good biocidal activity especially on staphylococcus aureus, very well-known as being responsible for infections spread in hospitals (nosocomial illnesses) is noted for the samples studied (glass plates and grafted fabric). After 48 hours colonies on the surface of the fabric and of the grafted film on glass are no being counted, after treatment and washing. The treated and washed fabric no longer has any antibacterial activity by diffusion, but it is very active by contact, owing to the grafted biocidal polymer, as the number of colonies is practically zero. Good efficacy by contact with the fungal growth after 48 hours is likewise proven (reduction of around 4 log).

1. A process for treating the surface of a solid substrate in which photopolymerisation and covalent grafting are performed in situ on said substrate of a biocidal copolymer, wherein the following steps are carried out in which:
a) said solid substrate is put in contact with a formulation comprising:
1—at least one monomer comprising a biocidal group,
2—at least one copolymerisable compound with said biocidal monomer comprising a mono-, di-, or plurifunctional monomer or oligomer selected amongst acrylate, epoxide and vinyl ether monomers or oligomers,
3—at least one photoprimer selected amongst radical and cationic photoprimers, and
4—at least one grafting agent on said substrate, and
b) photocopolymerisation and covalent grafting of the copolymers obtained are carried into effect by subjecting said formulation in contact with said solid substrate to ultraviolet radiation.

2. The process as claimed in claim 1, wherein in step a) for placing said formulation in contact with said substrate, the following two successive sub-steps are carried out:
a1) said solid substrate is put in contact with a first partial formulation containing said photoprimer and said grafting agent, and
a2) after drying, a second partial formulation containing said biocidal monomer and said copolymerisable compound is added.

3. The process as claimed in claim 1, wherein said biocidal monomer comprises a monomer comprising a group of quaternary salts responding to the formula (I) in which: 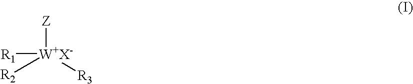
in which:
Z represents a radical monovalent selected amongst or either 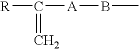
in which:
R represents —H or —CH₃
A represents 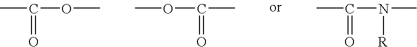
B represents an alkylene chain in C₁-C₅, linear or branched or an arylene or arylalkyelene group
either
CₙH₂ₙ₋₁(OH)₂—B—
or HO—(B—O)ₐ—B—
where n represents a whole number from 1 to 20, A represents a whole number from 0 to 3, and
B has the meaning given hereinabove
W⁺ represents a N⁺ nitrogen cation, a P⁺ phosphorous cation or a Q⁺ heterocycl cation, saturated or unsaturated, comprising a nitrogen atom substituted by R₃, or directly bonded to A or to B, and likewise able to contain in addition to quaternised nitrogen one or more heteroatoms, identical or different
R₁ and R₂ identical or different, each represent an alkyl chain in C₁-C₅ or an aryl group
R₃ represents an alkyl chain in C₃-C₂₀ or an aryl or arylalkyl group
X⁻ represents an anion.

4. The process as claimed in claim 3, wherein said biocidal monomer responds to the following formula (I₁): 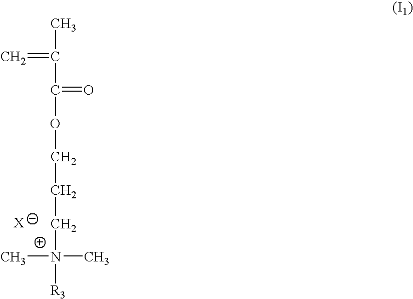
in which: R₃
R₃ represents an alkyl chain in C₃-C₂₀ an aryl or arylalkyl group
X⁻ represents an anion.

5. The process as claimed in claim 3, wherein said biocidal monomer responds to the following formula (I₂) 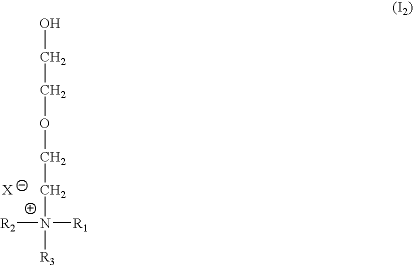
in which:
X⁻ represents an anion
R₁ and R₂ identical or different, each represent an alkyl chain in C₁-C₅ or an aryl group
R₃ represents an alkyl chain in C₃-C₂₀ or an aryl or arylalkyl group.

6. The process as claimed in claim 1, wherein said grafting agent comprises grafting primers preferably selected amongst the following compounds:
organic and inorganic peroxide compounds, optionally in a mixture with reductive organic compounds, especially amines, or in a mixture with metallic salts of Ag⁺, V²⁺, Ti²⁺, Co²⁺, Ce²⁺, Cu⁺, Fe²⁺, Na⁺, K^{+,} and
cerium and vanadium salts in their maximum oxidation state, Ce⁴⁺ and V⁵⁺.

7. The process as claimed in claim 1, wherein said grafting agent comprises a coupling agent preferably selected amongst:
compounds of silane type comprising (a) active groups photopolymerisable radically or cationic with said bactericidal monomers and said copolymerisable compounds and (b) groups enabling a covalent bond with groups of said substrate, and
compounds of organometallic salts.

8. The process as claimed in claim 1, wherein said copolymerisable compound comprises a mono or plurifunctional acrylate monomer or oligomer of general formula (II) 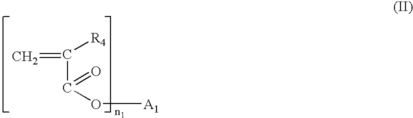
In which A₁ is an organic radical
R₄ is a hydrogen or a methyl
n₁ is a whole number from 1 to 6.

9. The process as claimed claim 1, wherein said copolymerisable compound comprises a mono, di or trifunctional epoxide monomer or oligomer, responding to the following general formula (III): 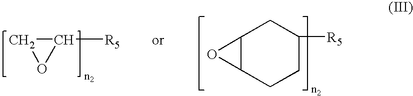
In which n₂ is a whole number from 1 to 3
R₅ is an organic radical.

10. The process as claimed in claim 1, wherein said copolymerisable compound comprises a vinyl ether monomer or oligomer responding to the following general formula (IV):
R₆—(O—CH═CH₂)_{1 or 2} (IV)
In which R₆ is an organic radical.

11. The process as claimed in claim 1, wherein said photoprimer comprises a radical photoprimer preferably comprising an organic compound containing at least one phenyl cycle substituted by a carbonyl, nitrogen, phosphorous or sulphur group.

12. The process as claimed in claim 1, wherein said photoprimer comprises a cationic photoprimer preferably selected amongst aryl sulfonium or aryl iodonium salts.

13. The process as claimed in claim 1, wherein the formulation comprises at least two photoprimers, respectively radical and cationic.

14. The process as claimed claim 1, wherein said formulation comprises its different constituents in the following weight proportions for a total of 100%, namely:
1) 5 to 95%, preferably 5 to 50%, of said biocidal monomers,
2) 5 to 95%, preferably 10 to 75%, of said copolymerisable compounds,
3) 1 to 10% of said photoprimer, and4) 0.01 to 10% of said grafting agent.

15. The process as claimed in claim 1, wherein in step 2), ultraviolet radiation is applied at an intensity of 10 to 5000 mW/cm², preferably from 100 to 1000 mW/cm², having a wavelength of between 280 and 500 nm, and a filter is preferably used effectively eliminating infrared radiation and irradiation having a wavelength of 360 to 500 nm.

16. The process as claimed in claim 1, wherein said substrate is constituted by a natural or synthetic organic material, preferably a material of plastic type, or a material based on natural polymer such as polysaccharides.

17. The process as claimed in claim 16, wherein said substrate is selected amongst fibrous textile or non-woven organic materials, based on synthetic or natural threads or fibres.

18. The process as claimed in claim 1, wherein said solid substrate is constituted by an inorganic material, preferably ceramic material or glass.

19. The process as claimed in claim 1, wherein said formulation comprises:
at least one grafting primer comprising preferably an organic peroxide compound or a cerium salt Ce⁴⁺, and
at least one said radical photoprimer.

20. The process as claimed in claim 1, wherein said formulation comprises:
at least one said cationic or radical photoprimer, and
at least one said coupling agent of silane type.

21. The process as claimed in claim 1 wherein said formulation comprises at least one said bi or pluri functional copolymerisable compound and said grafted biocidal copolymer obtained is reticulated.

22. A solid substrate comprising a polymer exhibiting biocidal properties, grafted to its surface, obtained by the process as claimed in claim 1.
